(19) United States
(12) Patent Application Publication
(10) Pub. No.: US 2006/0264637 A1
(43) Pub. Date: Nov. 23, 2006

(54) PREPARATION OF PAROXETINE HYDROCHLORIDE HEMIHYDRATE

(76) Inventors: Vijayavitthal Mathad Thippannachar, Hyderabad 500 070, A.P. (IN); Pravinchandra Vankawala Jayantilal, Hyderabad 500 049, A.P. (IN); Chandrasekhar Ravi Ram Elati, Hyderabad 500 072, A.P. (IN); Naveen Kumar Kolla, Hyderabad 500 072, A.P. (IN); Subrahmanyeswara Rao Chlamala, Hyderabad 500 072, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/383,306

(22) Filed: May 15, 2006

Related U.S. Application Data
(60) Provisional application No. 60/716,842, filed on Sep. 14, 2005.

(30) Foreign Application Priority Data
May 16, 2005 (IN) 578/CHE/2005

(51) Int. Cl. C07D 405/02

(52) U.S. Cl. 546/197

(57) ABSTRACT

A process for preparing paroxetine hydrochloride hemihydrate.

The present invention relates to an improved process for the preparation of paroxetine hydrochloride hemihydrate and intermediates thereof.

Paroxetine hydrochloride has the chemical name (−)-trans-4R-(4′-fluorophenyl)-3S-[(3′,4′-methylenedioxyphenoxy)methyl]piperidine hydrochloride and is represented by Formula I.

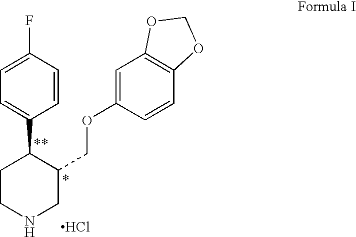

Paroxetine hydrochloride is a 5-hydroxytryptamine (5-HT, serotonin) re-uptake inhibitor and is useful as a psychotropic drug for its anti-depressant and anti-obsessional action. Pharmaceutical products containing paroxetine hydrochloride hemihydrate are commercially available in the market under the trademark PAXIL in various strengths.

U.S. Pat. No. 4,007,196 discloses paroxetine and a salt thereof with a pharmaceutically acceptable acid.

U.S. Pat. No. 4,721,723 discloses paroxetine hydrochloride hemihydrate and process for the preparation thereof.

U.S. Pat. No. 6,686,473 discloses an alternative process for the preparation of paroxetine by hydrolyzing paroxetine phenylcarbamate with a hydrolyzing agent in a solvent system comprising 1-butanol and a hydrocarbon cosolvent.

International Application Publication No. WO 2002/085360 A1 discloses a purification of the paroxetine free base compound of Formula III (below) by classical acid-base treatment and further conversion to the paroxetine hydrochloride hemihydrate compound of Formula I using toluene as a solvent.

EP 0223334 B1 and EP 0219934 B1 disclose a process for the preparation of the intermediate 4-(4-Fluorophenyl)piperidine-3-carboxylic acid methyl ester compound of Formula V (below) using platinum oxide as a hydrogenation catalyst for conversion from its preceding intermediate, the 4-(4-Fluorophenyl)-nicotinic acid methyl ester compound of Formula IV, and also disclose a preparation of the intermediate compound of Formula VIII using LiAIH₄ as a reducing agent for conversion from its preceding intermediate compound of Formula VII.

Further these EP patents also disclose the preparation of intermediate 4-(4-Fluorophenyl)-1-methylpiperidine-3-carboxylic acid methyl ester compound of Formula VI (below) by methylation of its preceding intermediate 4-(4-Fluorophenyl) piperidine-3-carboxylic acid methyl ester compound of Formula V, using expensive methylating agents such as methyl bromide in THF and methyl iodide.

Therefore, there is a need to provide a simple, improved, industrially feasible, economically inexpensive and safe to handle process using cost effective reagents for the synthesis of the compound of Formula I and intermediates thereof.

### SUMMARY OF THE INVENTION

The present invention relates to an improved process for the preparation of paroxetine hydrochloride hemihydrate and intermediates thereof.

Accordingly, the present invention in one aspect provides an improved process for the preparation of paroxetine hydrochloride hemihydrate comprising the steps of:

a) converting paroxetine phenylcarbamate intermediate compound of Formula II to the paroxetine freebase compound of Formula III;

b) reacting the paroxetine freebase compound of Formula III with hydrochloric acid to form paroxetine hydrochloride hemihydrate; and

c) optionally recrystallizing the compound in a suitable solvent to get pure paroxetine hydrochloride hemihydrate.

In another aspect the present invention provides a process for the preparation of the intermediate cis-(±)-[4-(4-Fluorophenyl)-1-methylpiperidin-3-yl]methanol compound of Formula VII comprising:

a) reduction of the intermediate 4-(4-Fluorophenyl)-nicotinic acid methyl ester compound of Formula IV using a hydrogenation catalyst to give the Cis-(±)-4-(4-Fluorophenyl)piperidine-3-carboxylic acid methyl ester compound of Formula V;

b) methylation of the intermediate compound of Formula V with a suitable methylating agent to give the Cis-(±)-4-(4-Fluorophenyl)-1-methyl-piperidine-3-carboxylic acid methyl ester compound of Formula VI; and

c) reduction of the compound of Formula VI using a suitable reagent in a suitable solvent to give the cis-(±)-[4-(4-Fluorophenyl)-1-methylpiperidin-3-yl]methanol compound of Formula VII.

In yet another aspect, the present invention provides paroxetine hydrochloride hemihydrate substantially free from one or more of the following impurities:

a) the (3S, 4R)-3-[(1,3-benzodioxol-5-yloxy)methyl-]-4-phenylpiperidine compound of Formula VIII (referred to as “Desfluoro Paroxetine”);

b) the (3S, 4R)-3-[(1,3-benzodioxol-5-yloxy)methyl-]-4-(4-methoxyphenyl) compound of Formula IX (referred to as “p- Methoxy Paroxetine”);

c) the (3S, 4R)-3-[(1,3-benzodioxol-5-yloxy)methyl-]-4-(4-ethoxyphenyl)piperidine compound of Formula X (referred to as “p-ethoxy Paroxetine”);

d) the (3S, 4R)-3-[(1,3-benzodioxol-5-yloxy)methyl-]-4-(4-flourophenyl)piperidine compound of Formula XI (referred to as “(+) Trans-paroxetine”);

e) the (3RS, 4RS)-3-[(1,3-benzodioxol-5-yloxy)methyl-]-4-(4-flourophenyl)piperidine compound of Formula XII (referred to as “(±) Cis-paroxetine”);

f) the 3,3′-{methylenebis (1,3-benzodioxole-6-4-diyloxymethylene)bis[4-(4-flourophenyl)piperidine compound of Formula XII (referred to as “Bis paroxetine”);

g) the 4-(4-flourophenyl)-1-methyl-1,2,3,6-tetra hydro pyridine compound of Formula XIV (referred to as “Tetra hydro pyridine”);

h) the (−) Trans-1-3-[1,3-benzodioxole-6-4-diyloxymethylene)bis[4-(4-flourophenyl)piperidine] compound of Formula XV (referred to as “N-methyl paroxetine”);

i) the (±) Trans 3-[(1,3-benzodioxol-5-yloxy)methyl]-4-(4″-fluorophenyl-4′-phenyl)piperidine HCl compound of Formula XVI;

j) the phenyl diethylcarbamate compound of Formula XVII;

k) the compound of Formula XVIII.

In yet another aspect, the invention comprises a process for preparing paroxetine hydrochloride hemihydrate, comprising:

adding sodium hydroxide to a refluxing solution comprising paroxetine phenyl carbamate and isopropanol, to form paroxetine free base;

reacting paroxetine free base with hydrochloric acid, in the presence of ethyl acetate, to form paroxetine hydrochloride hemihydrate; and

optionally recrystallizing in a system comprising acetone, water, and n-heptane to purify paroxetine hydrochloride hemihydrate.

A further aspect of the present invention provides paroxetine hydrochloride hemihydrate having a mean particle size not more than about 20 μm.

### DETAILED DESCRIPTION

The present invention relates to an improved process for the preparation of paroxetine hydrochloride hemihydrate and intermediates thereof.

The present invention in one aspect provides an improved process for the preparation of paroxetine hydrochloride hemihydrate comprising the steps of:

a) converting the paroxetine phenyl carbamate intermediate compound of Formula II; 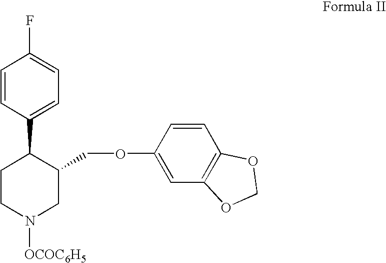
with a suitable base in a suitable solvent to form the paroxetine free base compound of Formula III;

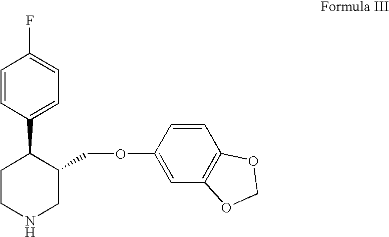

b) reacting the paroxetine free base compound of Formula III with hydrochloric acid in a suitable solvent to form paroxetine hydrochloride hemihydrate; and

c) optionally recrystallizing the compound in a suitable solvent to get pure paroxetine hydrochloride hemihydrate.

Step a) involves converting the paroxetine phenyl carbamate intermediate compound of Formula II to the paroxetine free base compound of Formula III by reacting with a suitable base in a suitable solvent.

Suitable solvents that can be used in the process of step a) include alcohols such as isopropanol, butanol, and the like, or water, or mixtures thereof.

Suitable inorganic bases that can be used in step a) include but are not limited to: hydroxides of alkali metals such as lithium hydroxide, sodium hydroxide, potassium hydroxide, and the like; carbonates of alkali metals such as sodium carbonate, potassium carbonate, and the like; and bicarbonates of alkali metals such as sodium bicarbonate, potassium bicarbonate, and the like; or mixtures thereof. Suitably the base can be added in solid form or it may be dissolved in the solvent and added in solution form.

Suitable temperatures for conducting the reaction can range from about 10° C. to 100° C., or about 30° C. to 90° C., or about 75° C. to 85° C.

Step b) involves reacting the paroxetine free base compound of Formula III with hydrochloric acid in a suitable solvent to form paroxetine hydrochloride hemihydrate.

Suitable solvents that can be used in the process of step b) include esters such as ethyl acetate, n-propyl acetate, n-butyl acetate, t-butyl acetate, and the like; and aromatic hydrocarbons such as toluene, xylene and the like; or mixtures thereof.

Hydrochloric acid that can be used in this process can be in the form of a methanolic hydrochloride, isopropanolic hydrochloride, ethyl acetate hydrochloride, aqueous hydrochloric acid, gaseous hydrochloric acid; and the like.

Suitable temperatures for conducting the reaction can range from about 10° C. to 100° C., or about 20° C. to 50° C., or about 25° C. to 35° C.

Step c) involves recrystallization of paroxetine hydrochloride obtained in step b) in a suitable solvent to give pure paroxetine hydrochloride hemihydrate, an embodiment comprising:

i) providing a suspension of paroxetine hydrochloride hemihydrate in a suitable solvent;

ii) adding water to the suspension of step i) at reflux to make a clear solution;

iii) adding an anti-solvent at room temperature;

iv) isolating paroxetine hydrochloride hemihydrate from the slurry.

The step of providing a solution of paroxetine hydrochloride hemihydrate may include dissolving any form of paroxetine hydrochloride hemihydrate in a suitable organic solvent or obtaining an existing solution from a previous processing step, such as a synthesis for paroxetine hydrochloride hemihydrate.

The suitable solvent that can be used in step i) includes ketones such as acetone, ethyl methyl ketone, methyl isobutyl ketone and the like; or mixtures thereof.

Suitable anti-solvents that can be used in step iii) include any solvent or mixture of solvents in which paroxetine hydrochloride hemihydrate has a limited solubility. Examples include: hydrocarbons such as n-hexane, n-heptane, cyclohexane and the like; ethers such as tetrahydrofuran, diethyl ether, diisopropyl ether, methyl tert-butyl ether, and the like; and mixtures thereof in any proportions.

The isolation of solid in step iv) can be carried out by using conventional techniques, such as centrifugation, decantation, gravity filtration, vacuum filtration, and the like.

The isolated solid product can optionally be dried using any technique, such as fluid bed drying (FBD), aerial drying, oven drying or other techniques known in the art at temperatures of about 25-100° C., or 50-60° C., with or without application of vacuum and/or under inert conditions.

In another aspect the present invention provides a process for the preparation of intermediate cis-(±)-[4-(4-Fluorophenyl)-1-methylpiperidin-3-yl]methanol, the compound of Formula VII, comprising:

a) reduction of the intermediate 4-(4-Fluorophenyl)-nicotinic acid methyl ester compound of Formula IV 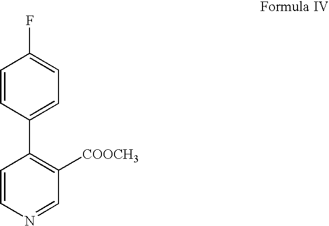
using a hydrogenation catalyst to give the Cis-(±)-4-(4-Fluorophenyl)piperidine-3-carboxylic acid methyl ester compound of Formula V;

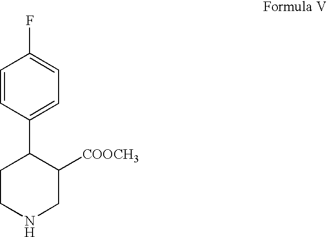

b) methylation of the intermediate compound of Formula V with a suitable methylating agent to give the Cis-(±)-4-(4-Fluorophenyl)-1-methyl-piperidine-3-carboxylic acid methylester compound of Formula VI; and

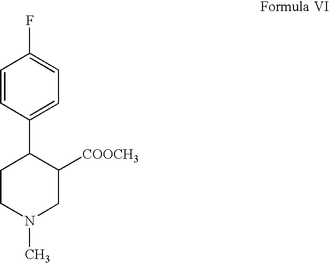

c) reduction of the compound of Formula VI using a suitable reagent in a suitable solvent to give the cis-(±)-[4-(4-Fluorophenyl)-1-methylpiperidin-3-yl]methanol compound of Formula VII.

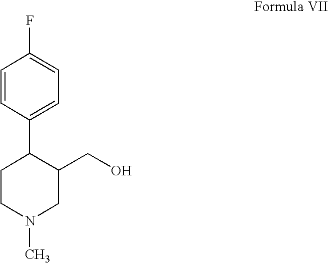

Step a) involves reduction of the intermediate 4-(4-Fluorophenyl)-nicotinic acid methyl ester compound of Formula IV using a hydrogenation catalyst to give the Cis-(±)-4-(4-Fluorophenyl)piperidine-3-carboxylic acid methyl ester compound of Formula V.

The suitable reducing agents include but are not limited to a noble metal catalyst such as nickel, palladium, iridium, ruthenium, and the like with hydrogen; or a transition metal catalyst in combination with HCl such as iron and HCl, zinc and HCl, tin and HCl, and the like.

The temperature for conducting the reaction can range from 30-100° C., or 50-80° C., or 60-70° C., and can use hydrogen gas pressures ranging from 2-8 kg/cm², or 4-5 kg/cm².

Step b) involves methylation of the intermediate compound of Formula V with a suitable methylating agent in a suitable solvent to give the Cis-(±)-4-(4-Fluorophenyl)-1-methyl-piperidine-3-carboxylic acid methylester compound of Formula VI;

Methylating agents that can be used in the process of step b) include but not limited to formaldehyde and formic acid, methyliodide, dimethylsulphate, dimethylcarbonate, and the like;

Suitable solvents include alcoholic solvents such as methanol, ethanol and the like; chloroform and the like; ketones such as acetone, ethyl methyl ketone and the like; esters such as ethyl acetate, n-propylacetate, n-butylacetate, t-butylacetate and the like; ethers such as diethylether, dimethylether, di-isopropylether, methyl tertiary-butyl ether and the like; hydrocarbons like toluene, xylene and the like; and mixtures thereof.

Step c) involves reduction of the compound of Formula VI using a suitable reducing reagent in a suitable solvent to give the cis-(±)-[4-(4-Fluorophenyl)-1-methylpiperidin-3-yl]methanol compound of Formula VII.

Suitable reducing agents that can be used in the process step c) include Vitride (having the chemical name sodium dihydro-bis-(2-methoxyethoxy)aluminate), sodium borohydride, and the like.

Suitable solvents that can be used include but are not limited to hydrocarbons such as toluene, xylene and the like; alcohols such as methanol, ethanol, isopropyl alcohol and the like; ketones such as acetone, ethyl methyl ketone and the like; esters such as ethyl acetate, n-propyl acetate, n-butyl acetate, t-butyl acetate and the like; ethers such as diethyl ether, dimethyl ether, di-isopropyl ether, methyl tertiary-butyl ether and the like; or mixtures thereof.

The intermediate compound of Formula VII may be converted into N-methyl paroxetine according to procedures described in U.S. Pat. No. 4,007,196 and may further be converted into the paroxetine phenyl carbamate intermediate compound of Formula II according to the process described in U.S. Pat. No. 6,686,473.

In a still further aspect, the present invention provides paroxetine hydrochloride hemihydrate substantially free from one or more of the following impurities:

a) the (3S,4R)-3-[(1,3-benzodioxol-5-yloxy)methyl-]-4-phenylpiperidine compound of Formula VIII (“Desfluoro Paroxetine”);

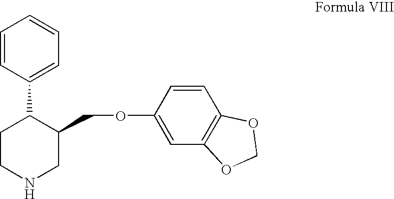

b) the (3S,4R)-3-[(1,3-benzodioxol-5-yloxy)methyl-]-4-(4-methoxyphenyl) compound of Formula IX (“p-Methoxy Paroxetine”);

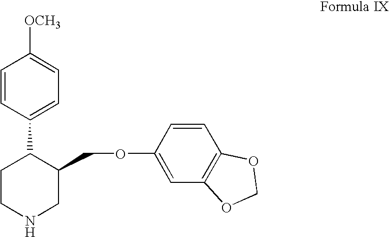

c) the (3S, 4R)-3-[(1,3-benzodioxol-5-yloxy)methyl-]-4-(4-ethoxyphenyl)piperidine compound of Formula X (“p-ethoxy Paroxetine”);

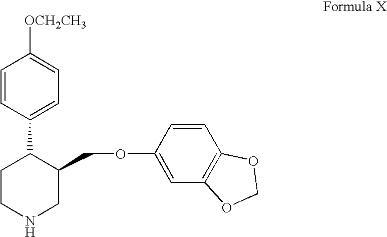

d) the (3S,4R)-3-[(1,3-benzodioxol-5-yloxy)methyl-]-4-(4-fluorophenyl)piperidine compound of Formula XI (“(±) Trans- paroxetine”);

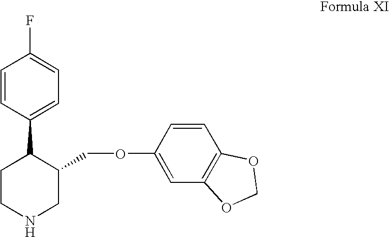

e) the (3RS,4RS)-3-[(1,3-benzodioxol-5-yloxy)methyl-]-4-(4-fluorophenyl)piperidine compound of Formula XII (“(±) Cis-paroxetine”);

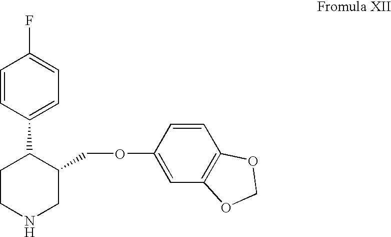

f) the 3,3′-{methylenebis (1,3-benzodioxole-6-4-diyloxymethylene)bis[4-(4-fluorophenyl)piperidine compound of Formula XII (“Bis paroxetine”);

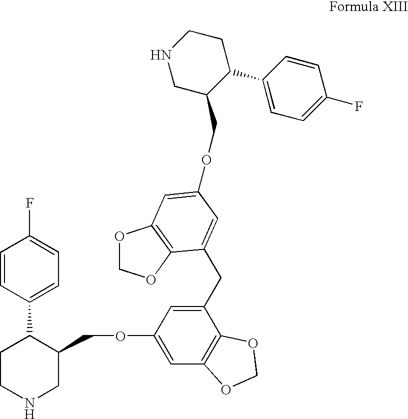

g) the 4-(4-flourophenyl)-1-methyl-1,2,3,6-tetra hydro pyridine compound of Formula XIV (“Tetra hydro pyridine”);

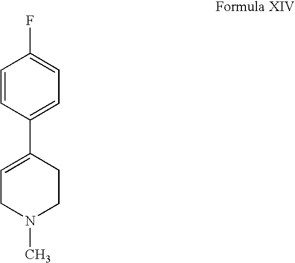

h) the (−) Trans -1-3-[1,3-benzodioxole-6-4-diyloxymethylene)bis[4-(4-fluorophenyl)piperidine] compound of Formula XV (“N-methyl paroxetine”);

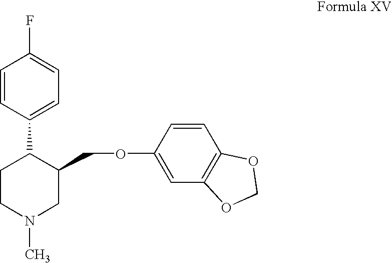

i) the (±) Trans 3-[(1,3-benzodioxol-5-yloxy)methyl]-4-(4″-fluorophenyl-4′-phenyl)piperidine HCl compound of Formula XVI;

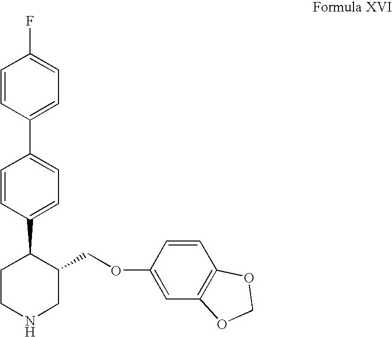

j) the phenyl diethylcarbamate compound of Formula XVII; and

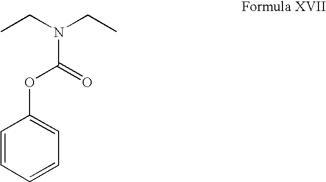

k) the compound of Formula XVIII.

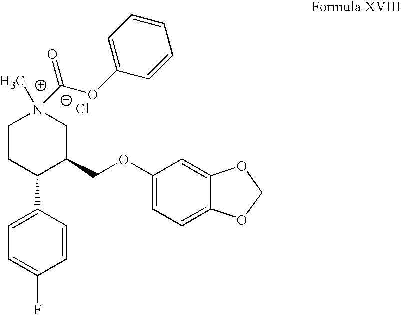

As used herein, the term “substantially free” refers to paroxetine hydrochloride hemihydrate having no greater than about 0.15 percent, or about 0.10 percent, of one or more of the aforementioned impurities, as determined by HPLC.

In one embodiment paroxetine hydrochloride hemihydrate of the present invention contains not more than 5 ppm, or not more than 1 ppm, of the tetrahydro pyridine impurity compound of Formula XIV when analyzed by HPLC (Method I) performed using an ACE-C18 column (250 cm×4.0 mm, 5 μm particle size) with the following parameters.
Detector: Variable wavelength UV-detector
Wavelength: 242 nm
Injection volume: 75μl
Flow rate: 1.5 ml/minute
Column oven temperature: 30° C.
Run time: 45 minutes
Mobile phase A: 30 g of sodium perchlorate in about 900 ml of water and added 3.5 ml of phosphoric acid and 2.4 ml of triethylamine. Diluted the contents to 1000 ml and pH was adjusted to about 2.0 with phosphoric acid and triethyl amine
Mobile phase B: Filtered and degassed Acetonitrile

Gradient program:

Time (min)

| | 0.01 | 20.0 | 27.0 | 36.0 | 38.0 | 45.0 |
|---|---|---|---|---|---|---|
| % B | 15 | 20 | 45 | 45 | 20 | 15 |

In a further embodiment paroxetine hydrochloride hemihydrate of the present invention contains not more than 0.5 percent, or not more than 0.1 percent, of the (±)-trans paroxetine impurity compound of Formula XI when analyzed by chiral HPLC (Method II) performed using a Chiralpak ADH column (250 cm×4.6 mm, 5 μm particle size) with the following parameters.
Detector: Variable wavelength UV-detector
Wavelength: 295 nm
Injection volume: 10
Flow rate:1.0 ml/minute
Column oven temperature: 30° C.
Run time: 50 minutes
Mobile phase: Mixture of n-hexane, 2-propanol and diethyl amine in the ratio of 96:4:0.3

All other impurities except compounds of Formula XIV and Formula XI can be analyzed by HPLC (Method III) performed using an Inertsil C8-3 column (250 cm×4.6 mm, 5 μm particle size) with the following parameters.
Detector: Variable wavelength UV-detector
Wavelength: 285 nm
Injection volume: 20μl
Flow rate: 1 ml/minute
Column oven temperature: 35° C.
Run time: 70 minutes
Mobile phase A: A filtered and degassed mixture of water, THF and trifuloroacetic acid in the ration of 90:10:0.5
Mobile phase B: A filtered and degassed mixture of Acetonitrile, THF and trifuloroacetic acid in the ratio of 90:10:0.5

Gradient program:

Time (min)

| | 0.01 | 30.0 | 50.0 | 60.0 | 65.0 | 70.0 |
|---|---|---|---|---|---|---|
| % B | 20 | 20 | 80 | 80 | 20 | 20 |

Paroxetine hydrochloride hemihydrate prepared in this process contains residual solvents, etc. at concentrations equal to or less than the requirements of ICH guidelines when analyzed by gas chromatography. For example paroxetine hydrochloride hemihydrate prepared according to this process contains: acetone at less than about 5000 ppm, or less than about 1000 ppm, or less than about 100 ppm; methanol at less than about 3000 ppm, or less than about 1000 ppm, or less than about 100 ppm; toluene at less than about 890 ppm, or less than about 200 ppm, or less than about 50 ppm; ethyl acetate at less than about 5000 ppm, or less than about 1000 ppm, or less than about 100 ppm; diethyl ether at less than about 5000 ppm, or less than about 100 ppm, or less than about 50 ppm; n-heptane at less than about 5000 ppm, or less than about 1000 ppm, or less than about 50 ppm; isopropanol at less than about 5000 ppm, or less than about 100 ppm, or less than about 50 ppm; ethanol at less than about 5000 ppm, or less than about 1000 ppm, or less than about 50 ppm; and acetic acid at less than about 5000 ppm, or less than about 1000 ppm, or less than about 50 ppm In yet another aspect, the present invention provides paroxetine hydrochloride hemihydrate having a mean particle size not more than about 20 μm.

The D₁₀, D₅₀ and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀ and D₉₀ include laser diffraction, such as using Malvern Instruments Ltd. (of Malvern, Worcestershire, United Kingdom) equipment.

In an embodiment, paroxetine hydrochloride hemihydrate according to the invention has a D₁₀ less than about 20 μm or less than about 10 μm, D₅₀ less than about 40 μm or less than about 20 μm, and D₉₀ less than about 100 μm or less than about 50 μm. There is no specific lower limit for any of the D values.

The invention is further described by reference to the following examples, which set forth in detail certain specific aspects and embodiments of the present invention. It will be apparent to those skilled in the art that many modifications, both to materials and methods, can be practiced without departing from the purpose and intent of this invention. The examples that follow are not intended to limit the scope of the invention as described hereinabove or as claimed below.

### EXAMPLE 1

### Preparation of paroxetine phenylcarbamate

14 kg of N-methyl paroxetine was charged into a clean, dry reactor containing 105 L of toluene at 27° C. The contents were heated to about reflux and 11.5 kg of phenyl chloroformate was added over about 40 minutes at 105-108° C. The reaction mass was maintained for about 3 hours at reflux and reaction completion was confirmed by thin layer chromatography (TLC). The reaction mixture was cooled to about 60° C. and 4.2 kg of triethylamine was added in 15 minutes. The reaction mixture was maintained for about 1 hour at 60-62° C. The reaction mixture was cooled to about 30° C. and 30.8 L of 5% aqueous sodium hydroxide solution was added. The reaction mixture was stirred for about 20 minutes. The organic layer was separated and washed with 28 L of 10% aqueous sodium chloride solution. The organic layer was separated and 28 L water was added. The reaction mass pH was adjusted to 2.0 with 36% aqueous hydrochloric acid solution (1 L) and stirred for about 15 minutes. Separated the organic layer and washed with of water (3×28 L). Final organic layer was separated (Volume: 105 L) and 75 L of the solvent was distilled off at about 60° C. under a vacuum of about 640 mm Hg. 112 L of methanol was charged slowly to the mass and stirred at about 62° C. for about 20 minutes to form clear solution. The reaction mass was cooled to about 30° C. and stirred for about 30 minutes. The reaction mass was further cooled to 2° C. and stirred for about 4 hours at 0-4° C. The reaction mass was centrifuged and wet solid was washed with 14 L of chilled methanol. The solid was dried at about 52° C. under a vacuum of about 600 mm Hg for about 3 hours to get 16.86 kg of the title compound

### EXAMPLE 2

### Preparation of paroxetine Free Base

16 kg of paroxetine phenylcarbamate of example 1 was charged into a clean, dry reactor containing 160 L of isopropyl alcohol at 27° C. The contents were heated to about 79° C. and aqueous sodium hydroxide solution (prepared by dissolving 17.13 Kg of sodium hydroxide flakes in 48 L of water) was added over about 1 hour, 20 minutes. The reaction mass was maintained for about 4 hours at reflux and reaction completion was confirmed by thin layer chromatography (TLC). The reaction mixture was cooled to about 35° C. and 112 L of water was charged. The reaction mixture was extracted with toluene (3×48 L) and the organic layer was washed with 48 L of water. Organic layer was separated and washed with of 5% aqueous sodium hydroxide solution (4×48 L). Separated the organic layer and washed with water (5×32 L). Final organic layer was separated and the solvent distilled completely at about 60° C. under a vacuum of about 700 mm Hg to yield 11.58 kg of free base compound.

### EXAMPLE 3

### Preparation of paroxetine hydrochloride hemihydrate

11.58 kg of paroxetine free base was charged into a reactor containing 81.06 L of ethyl acetate and stirred for about 15 minutes at about 30° C. to form a clear solution. The reaction mass was filtered through a filtration system containing an online filter (5 micron polypropylene cloth), a 0.45 micron cartridge polypropylene filter, and a 0.2 micron cartridge polypropylene filter, and the filtration system was washed with 34.74 L of ethyl acetate. 2.895 L of 36% aqueous hydrochloric acid was added slowly to the filtrate over about 30 minutes at about 28-33° C. The reaction mass was stirred for about 1 hour, 45 minutes at about 28-30° C. It was then cooled to about 1° C. and maintained for about 1 hour, 30 minutes at about 0-2° C. The reaction mass was centrifuged and the wet cake washed with 23.16 L of chilled ethyl acetate. The wet solid was dried at 30° C. for about 1 hour, 15 minutes under a vacuum of about 690 mm Hg. The solid was further dried at about 58° C. under a vacuum of about 690 mm Hg for about 4 hours to get 10.9 kg of the paroxetine hydrochloride.

### EXAMPLE 4

### Purification of paroxetine hydrochloride hemihydrate

10.9 kg of the paroxetine hydrochloride obtained in Example 3 above was charged into a clean, dry reactor containing 76.3 L of acetone and the contents were heated to reflux. 4.564 L of water was added to the reaction suspension at reflux for about 60 minutes and stirred at reflux for about 20 minutes to form a clear solution. The reaction mass was cooled slowly to about 33° C. in 2 hours and then stirred for 1 hour at about 30-33° C. 32.7 L of n-heptane was charged into the reactor and stirring was continued for about 1 hour, 30 minutes. The reaction mass was cooled to about 2° C. and stirred for about 2 hours. The reaction mass was centrifuged and wet cake was washed with 6.54 L of chilled acetone. The wet solid was dried under vacuum of about 690 mm Hg at about 30° C. for about 2 hours and then at about 55-57° C. for about 4 hours. The resultant solid was milled in a micronizer (Manufacturer: Microtech Engineering company, Model: M-50) with an air pressure of 0.5 kg/cm² at a feed rate of 7 kg/hour and then sieved through a 10 mesh sieve yielding 8.7 Kg of paroxetine hydrochloride hemihydrate with particle size distribution: D₁₀=2.37 μm; D₅₀=11.1 μm; and D₉₀=30.6 μm.

### EXAMPLE 5

### Preparation of 4-(4-fluorophenyl)piperidine-3-carboxylic acid methyl ester (Formula V)

200 g of 4-(4-Fluorophenyl)-nicotinic acid methyl ester, 800 ml of glacial acetic acid, 200 g of 5% palladium supported on carbon, and 800 ml of methanol were charged into an autoclave vessel at about 27° C. Dry hydrogen gas pressure of about 5 kg/cm² was applied to the reaction suspension and the contents were heated to about 70° C. The reaction mass was maintained under agitation for about 3 hours with a hydrogen gas pressure of about 5 kg/cm² and a temperature of about 70° C. Reaction suspension was cooled to about 20° C., the catalyst was removed by filtration through a flux calcined diatomaceous earth (Hyflow) bed and the bed washed with 400 ml of methanol. Solvent was distilled completely in a Buchi Rotovapor at about 45-52° C. under vacuum, and the residue was cooled to about 26° C. The resultant residue was dissolved in 2 L of water and the solution washed with toluene (2×1 L). The aqueous layer was separated and pH was adjusted to about 11.5 with 50% aqueous sodium hydroxide solution. The aqueous layer was extracted with toluene (3×500 ml) and the toluene organic layer was washed with water (2×500ml). Separated the organic layer and dried with sodium sulphate (100 g). The organic layer was distilled completely in a Buchi Rotovapor at about 55° C. under vacuum to yield 138 g. of the title compound as a residue.

### EXAMPLE 6

### Preparation of 4-(4-fluorophenyl)-1-methyl-piperidine-3-carboxylic acid methyl ester (Formula VI)

132 g of 4-(4-Fluorophenyl)piperidine-3-carboxylic acid methyl ester, water (1320 ml), formaldehyde (74.9 ml of 37% aqueous solution), and formic acid (52.8 ml) were charged in a clean 4-neck round bottom flask. The reaction mixture was heated to about 98° C. and maintained under stirring for about 2 hours, 30 minutes. The reaction mass was cooled to about 25° C. and washed with toluene (2×660 ml). Reaction mass pH was adjusted to about 10.5 with 50% aqueous sodium hydroxide solution and the resultant reaction solution was extracted with toluene (2×1320 ml). The toluene organic layer was separated and washed with water (2×660 ml). Final organic layer was separated and the solvent distilled completely at about 55° C. under vacuum to yield 122.7 g. of the title compound as residue.

### EXAMPLE 7

### Preparation of [4-(4-fluorophenyl)-1- methylpiperidin-3-yl]methanol (Formula VII)

2.0 g of 4-(4-Fluorophenyl)-1-methyl-piperidine-3-carboxylic acid methyl ester was charged into a 4-neck round bottom flask containing 20 ml of toluene under a nitrogen atmosphere at 27° C. 5 ml of Vitride (65% solution of sodium dihydro-bis-(2-methoxyethoxy)aluminate in toluene) was added slowly over about 15 minutes at below 40° C. The reaction mixture was cooled to about 12° C. and quenched by the addition of a mixture of water (10 ml) and 36% aqueous hydrochloric acid (10 ml) over about 15 minutes. Aqueous layer was separated and extracted with 20 ml toluene. Total organic layer was washed with water (2×20 ml) and the solvent was distilled completely in a Buchi Rotovapor at about 55° C. under vacuum to yield 1.3 g of the title compund as a residue.

We claim:

1. A process for preparing paroxetine hydrochloride hemihydrate, comprising:
reacting paroxetine phenyl carbamate with a basic compound to form paroxetine free base;
reacting paroxetine free base with hydrochloric acid to form paroxetine hydrochloride hemihydrate; and
optionally recrystallizing to purify paroxetine hydrochloride hemihydrate.

2. The process of claim 1, wherein a basic compound comprises at least one alkali metal hydroxide, carbonate, or bicarbonate.

3. The process of claim 1, wherein a basic compound comprises sodium hydroxide.

4. The process of claim 1, wherein reacting paroxetine phenyl carbamate with a basic compound is conducted in a solvent comprising isopropanol.

5. The process of claim 1, wherein reacting paroxetine phenyl carbamate with a basic compound comprises adding sodium hydroxide to a refluxing solution.

6. The process of claim 1, wherein reacting paroxetine free base with hydrochloric acid is conducted in a solvent comprising ethyl acetate.

7. The process of claim 1, wherein recrystallizing is conducted in a system comprising acetone, water, and n-heptane.

8. The process of claim 1, wherein recrystallizing comprises adding water to a refluxing acetone solution of paroxetine hydrochloride hemihydrate.

9. The process of claim 1, wherein paroxetine hydrochloride hemihydrate has a mean particle size not more than about 20 μm.

10. The process of claim 1, wherein the paroxetine hydrochloride hemihydrate contains less than about 0.15 percent of any one or more of the impurities:
a) (3S,4R)-3-[(1,3-benzodioxol-5-yloxy)methyl-]-4-phenylpiperidine of Formula VIII; 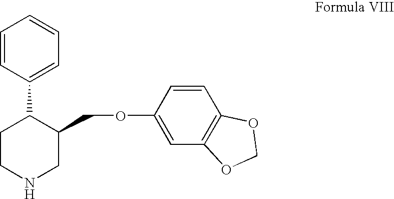
b) (3S,4R)-3-[(1,3-benzodioxol-5-yloxy)methyl-]-4-(4-methoxyphenyl) of Formula IX; 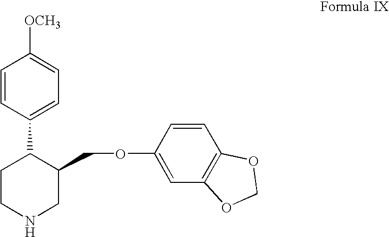
c) (3S, 4R)-3-[(1,3-benzodioxol-5-yloxy)methyl-]-4-(4-ethoxyphenyl)piperidine of Formula X; 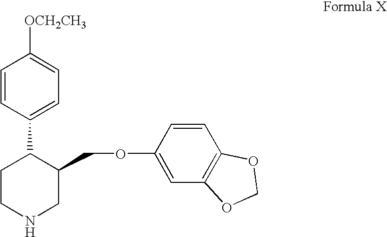
d) (3S,4R)-3-[(1,3-benzodioxol-5-yloxy)methyl-]-4-(4-fluorophenyl)piperidine of Formula XI; 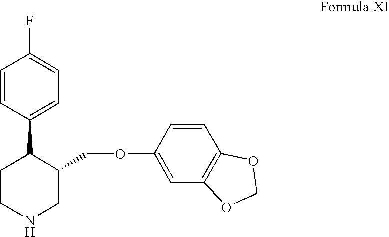
e) (3RS,4RS)-3-[(1,3-benzodioxol-5-yloxy)methyl-]-4-(4-fluorophenyl)piperidine of Formula XII; 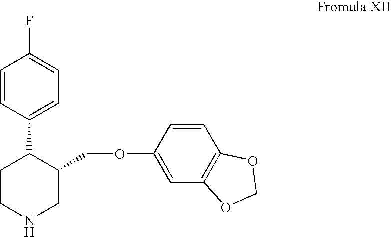
f) 3,3′-{methylenebis (1,3-benzodioxole-6-4-diyloxymethylene)bis[4-(4-fluorophenyl)piperidine of Formula XIII; 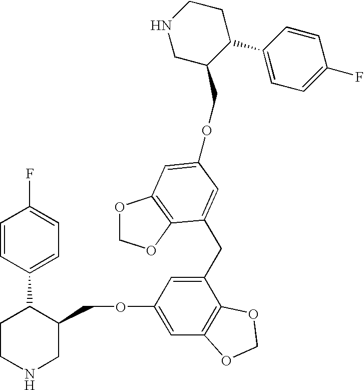
Formula XIII
g) 4-(4-fluorophenyl)-1-methyl-1,2,3,6-tetra hydro pyridine of Formula XIV; 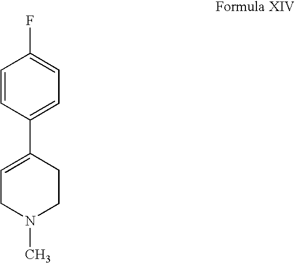
h) (−) Trans -1-3-[1,3-benzodioxole-6-4-diyloxymethylene)bis[4-(4-fluorophenyl)piperidine] of Formula XV; 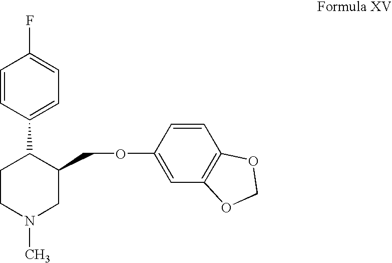
i) (±) Trans 3-[(1,3-benzodioxol-5-yloxy)methyl]-4-(4″-fluorophenyl-4′-phenyl)piperidine HCl of Formula XVI; 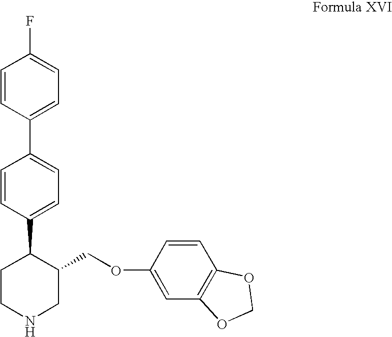
j) phenyl diethylcarbamate of Formula XVII; and 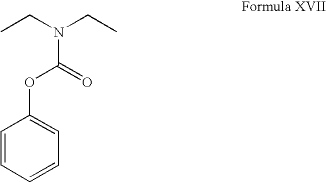
k) a compound of Formula XVIII

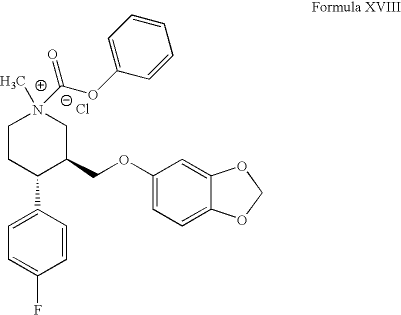

11. The process of claim 1, wherein the paroxetine hydrochloride hemihydrate contains less than about 5 ppm by HPLC of 4-(4-fluorophenyl)-1-methyl-1,2,3,6-tetra hydro pyridine of Formula XIV

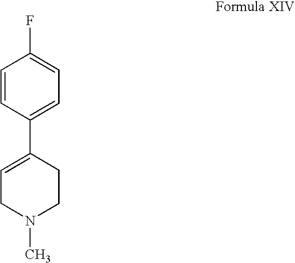

12. The process of claim 1, wherein the paroxetine hydrochloride hemihydrate contains less than about 0.5 percent by HPLC of (3S,4R)-3-[(1,3-benzodioxol-5-yloxy)methyl-]-4-(4-fluorophenyl)piperidine of Formula XI

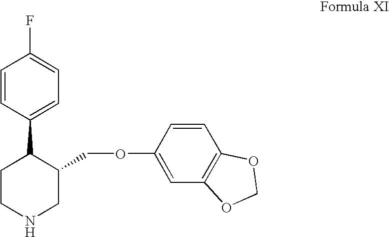

13. A process for preparing cis-(±)-[4-(4-Fluorophenyl)-1-methylpiperidin-3-yl]methanol, comprising:
a) hydrogenating 4-(4-Fluorophenyl)-nicotinic acid methyl ester of Formula IV 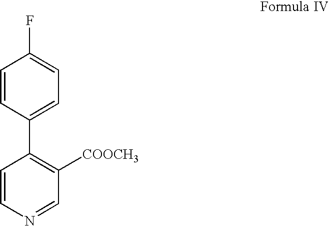
to form Cis-(±)-4-(4-Fluorophenyl)piperidine-3-carboxylic acid methyl ester of Formula V; 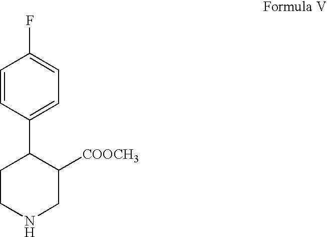
b) methylating the compound of Formula V to give Cis-(±)-4-(4-Fluorophenyl)-1-methyl-piperidine-3-carboxylic acid methylester of Formula VI; and 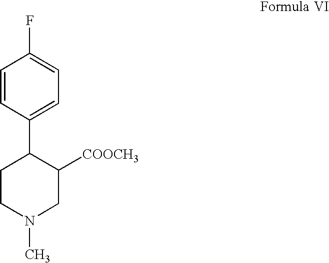
c) reducing the compound of Formula VI to give cis-(±)-[4-(4-Fluorophenyl)-1-methylpiperidin-3-yl]methanol of Formula VII.

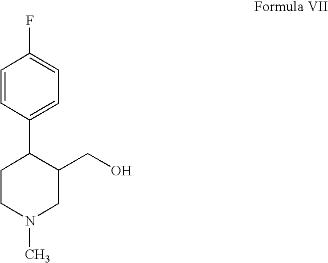

14. The process of claim 13, wherein hydrogenating is conducted in the presence of a catalyst comprising palladium.

15. The process of claim 13, wherein methylating is conducted with reagents comprising formaldehyde and formic acid.

16. The process of claim 13, wherein reducing is conducted by reacting with sodium dihydro-bis-(2-methoxyethoxy) aluminate.

17. A process for preparing paroxetine hydrochloride hemihydrate, comprising:
adding sodium hydroxide to a refluxing solution comprising paroxetine phenyl carbamate and isopropanol, to form paroxetine free base;
reacting paroxetine free base with hydrochloric acid, in the presence of ethyl acetate, to form paroxetine hydrochloride hemihydrate; and
optionally recrystallizing in a system comprising acetone, water, and n-heptane to purify paroxetine hydrochloride hemihydrate.

18. The process of claim 17, wherein recrystallizing comprises adding water to a refluxing acetone solution of paroxetine hydrochloride hemihydrate.

19. The process of claim 17, wherin the paroxetine hydrochloride hemihydrate contains less than about 0.15 percent of any one or more of the impurities:
a) (3S,4R)-3-[(1,3-benzodioxol-5-yloxy)methyl-]-4-phenylpiperidine of Formula VIII; 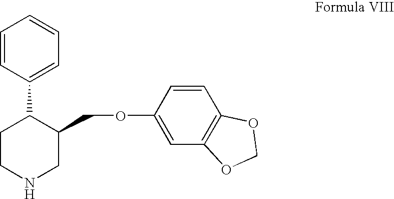
b) (3S,4R)-3-[(1,3-benzodioxol-5-yloxy)methyl-]-4-(4-methoxyphenyl) of Formula IX; 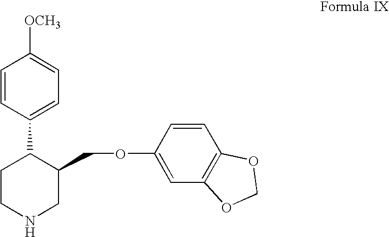
c) (3S, 4R)-3-[(1,3-benzodioxol-5-yloxy)methyl-]-4-(4-ethoxyphenyl) piperidine of Formula X; 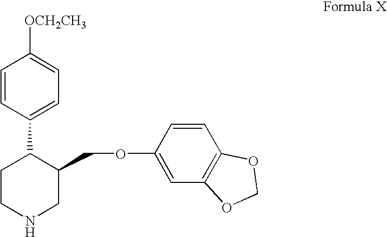
d) (3S,4R)-3-[(1,3-benzodioxol-5-yloxy)methyl-]-4-(4-fluorophenyl)piperidine of Formula XI; 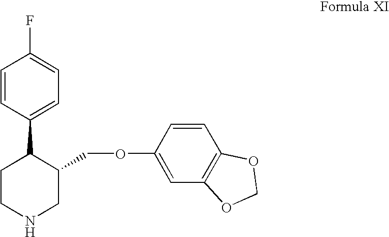
e) (3RS,4RS)-3-[(1,3-benzodioxol-5-yloxy)methyl-]-4-(4-fluorophenyl)piperidine of Formula XII; 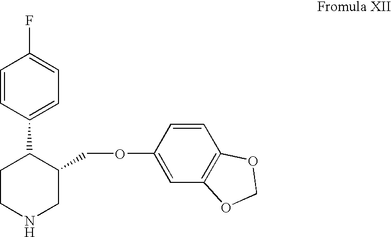
f) 3,3′-{methylenebis (1,3-benzodioxole-6-4-diyloxymethylene)bis [4-(4-fluorophenyl)piperidine of Formula XIII; 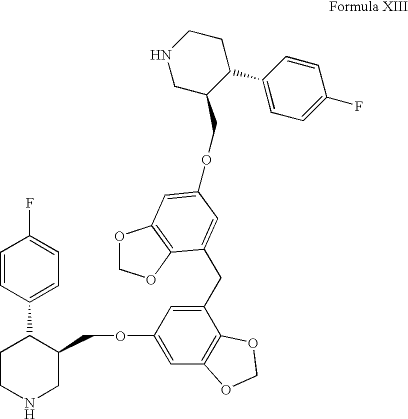
g) 4-(4-fluorophenyl)-1-methyl-1,2,3,6-tetra hydro pyridine of Formula XIV; 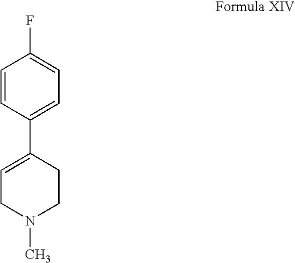
h) (−)-Trans -1-3-[1,3-benzodioxole-6-4-diyloxymethylene)bis[4-(4-fluorophenyl)piperidine] of Formula XV; 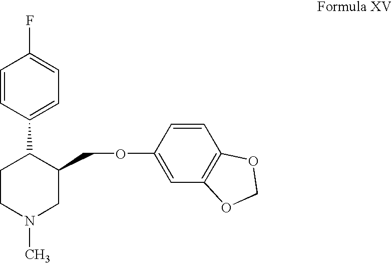
i) (±) Trans 3-[(1,3-benzodioxol-5-yloxy)methyl]-4-(4″-fluorophenyl-4′-phenyl)piperidine HCl of Formula XVI; 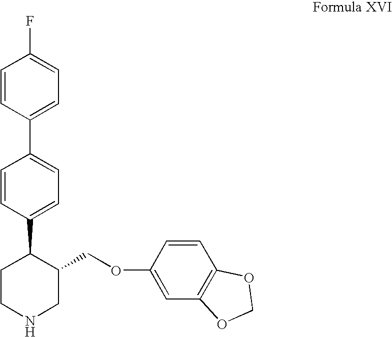
j) phenyl diethylcarbamate of Formula XVII; and 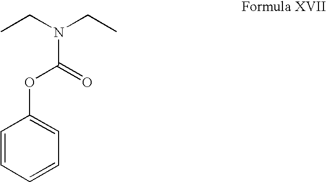
k) a compound of Formula XVIII

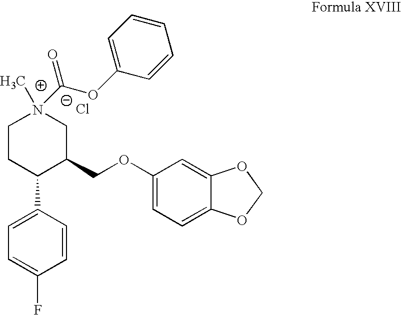

20. The process of claim 17, wherein the paroxetine hydrochloride hemihydrate contains less than about 5 ppm by HPLC of 4-(4-fluorophenyl)-1-methyl-1,2,3,6-tetra hydro pyridine of Formula XIV 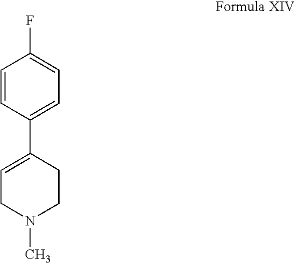
and less than about 0.5 percent by HPLC of (3S,4R)-3-[(1,3-benzodioxol-5-yloxy)methyl-]-4-(4-fluorophenyl)piperidine of Formula XI [chemical structure]
